(19) United States
(12) Patent Application Publication
(10) Pub. No.: US 2026/0115392 A1
(43) Pub. Date: Apr. 30, 2026

(54) PULMONARY DRUG DELIVERY APPARATUS

(71) Applicant: INSPIRING HOLDINGS PTY LTD, Dalkeith (AU)

(72) Inventor: Barry Spencer Clements, Dalkeith (AU)

(21) Appl. No.: 19/470,690

(22) PCT Filed: Apr. 20, 2024
(86) PCT No.: PCT/AU2024/050380

§ 371 Date: Sep. 29, 2025

(30) Foreign Application Priority Data
Apr. 20, 2023 (AU) 2023901167

(51) Int. Cl. A61M 15/00

(52) U.S. Cl. CPC A61M 15/002; A61M 15/009; A61M 2205/02; A61M 2205/581; A61M 2230/42

(57) ABSTRACT

Pulmonary drug delivery apparatus (10) comprising a spherical reservoir (12) defining respective inlet and outlet openings (14, 16) through a surface (18) thereof, and an elongate, arcuate and substantially flat spine member (20) defining an inlet aperture (22) therethrough at one end and an outlet aperture (24) therethrough at another end. The spine member (20) is configured to engage complementarily about an outer surface portion of said spherical reservoir (12) such that i) the inlet aperture (22) cooperates with the inlet opening (14) to form an inlet port (26) normal to the surface (18) of the spherical reservoir (12); and ii) the outlet aperture (24) cooperates with the outlet opening (16) to define an outlet port (28) tangentially arranged on the surface (18) of the spherical reservoir (12). In this manner, the inlet and outlet ports (26 and 28) are opposed and out of alignment on the spherical reservoir (12).

### TECHNICAL FIELD

This invention relates broadly to the field of pulmonary drug delivery, and more specifically to pulmonary drug delivery apparatus.

### BACKGROUND ART

The following discussion of the background art is intended to facilitate an understanding of the present invention only. The discussion is not an acknowledgement or admission that any of the material referred to is or was part of the common general knowledge as at the priority date of the application.

Pulmonary drug delivery is a route of administration in which patients use an inhalation drug delivery device, such as an inhaler, to inhale their medications and drugs to act on a target site in the airway, or even for absorption into the bloodstream via the lung mucous membrane. This technique is most commonly used in the treatment of lung diseases, for example, asthma and chronic obstructive pulmonary disease (COPD). Different types of inhalers include metered-dose inhalers (MDI), dry powder inhalers (DPI) and nebulisers.

Proper education on inhaler use is important to ensure that inhaled medication is effectively delivered to the airway to create its proper effects. The rate, efficiency and efficacy of pulmonary drug delivery are affected by drug particle properties, breathing patterns and respiratory tract geometry. As a result, a rate, efficiency and efficacy of pulmonary drug delivery are affected by drug particle properties, breathing patterns and respiratory tract geometry, typically making such pulmonary drug administration dependent on inhaler techniques and a patients'compliance with proper use of an inhaler, i.e. poor timing, coordination and breathing depth, rate and pattern.

To achieve successful pulmonary drug delivery, inhaled particles should ideally not deposit on the upper respiratory tract since they will be swallowed or expectorated without reaching the lungs, leading to the loss of intended pharmacological effect and/or provoking unwanted systemic side effects. Incorrect inhaler techniques, such as poor coordination, no exhalation before inhaling the drug aerosol or not holding breath for a few seconds after inhalation may lead to medication depositing inside the respiratory tract instead of the lungs, resulting in inefficient and inadequate treatment.

Different inhalers require specific technique to use properly, for example metered-dose inhalers (MDIS) require coordination between inhalation and inhaler actuation, dry powder inhalers (DPIs) require adequate inspiratory flow from patients and can be moisture sensitive, and nebulisers may provide low drug delivery efficiency to the lungs, as a substantial percentage of medication is typically lost to the atmosphere during use, which can cause a hazard to others around the patient.

In an attempt to address some of these shortcomings, the art turned to inhaler spacers designed for use with an MDI only, which are generally some manner of device which provides a space typically in the form of a tube or chamber between the mouth of a patient and an MDI. Such spacers are intended to assist patients with inhaling their medication more effectively by introducing a storage space where medication can be stored until the patient is ready to inhale. The volume of the spacer where the medication is stored is important with small volume spacers (which constitute over 80% of spacers sold) only minimally reducing the requirement to synchronise a patient's breathing so that they can inhale properly to facilitate pulmonary drug administration. The walls of conventional spacers are typically made of rigid plastic where droplets impact and are retained, reducing the amount of drug exiting the spacer into the patient's mouth by up to 40%. This reduces the amount of drug reaching the airway to produce its effect, substantially limiting the intended benefits of the spacer, particularly in children, people with severe shortness of breath, and those with cognitive impairment. The general bulkiness of spacers limits their appeal with patients.

A variety of spacers have been developed in the art. For example, US 2016/0256641 to Lisberg describes a device which includes a collapsible bag to which is attached a bidirectional mouthpiece and an adaptor that receives an MDI. The mouthpiece contains a reed that functions as an audible signal and a screen to prevent inhalation of unwanted particles. U.S. Pat. No. 4,790,305 to Zoltan et al. teaches apparatus comprising a mouthpiece for supplying a pharmaceutical composition to the mouth of a patient, a rigid chamber for holding the aerosol prior to inhalation, the rigid chamber having orifices to limit the flow of air therethrough, and a collapsible chamber from which the patient inhales unmedicated air prior to inhaling the medicated air from the rigid chamber.

U.S. Pat. No. 4,484,577 to sackner discloses apparatus which includes an expanded bag and bidirectional channel for communicating a drug with the breathing passage of a patient and which is substantially impervious to the passage of air. Collapsing the expanded bag while the bidirectional channel for communicating with the breathing passage is in position delivers the drug into the breathing passage of the patient, with a signal in the bidirectional channel indicates when the rate of passage of the drug exceeds a desirable limit.

U.S. Pat. No. 7,418,962 to Rao describes an inhaler having a collapsible recirculation chamber or rebreather. The inhaler has an aerosol holding chamber having an inlet end for attaching a holder for aerosol medication or nebulizer device thereto and an outlet end for attachment to a mouthpiece or mask for dispensing the medication or substance to the user, with a recirculation chamber depending from the outlet end of the holding chamber or from a mask extending from the outlet end of the chamber.

U.S. Pat. No. 5,791,340 to Schleufe et al. teaches a resuscitator for artificial resuscitation comprising a bag which, at its one end is provided with a patient valve, and at its opposite end is provided with an inlet valve for drawing in fresh ambient air. Between the surroundings and the bag interior, a small flow-through opening is provided, through which the bag may be supplied with the contents of a dosage aerosol for administration by inhalation, of pharmaceutically active air cells, allowing use of the resuscitator as an inhalation spacer.

U.S. Pat. No. 7,360,537 to Snyder at al. discloses aerosol medication delivery apparatus including an antistatic holding chamber having an input end and an output end and defining an interior space. GB 2285396 to Savoullas describes an inhaler comprising a suspended rebreathing bag, the inhaler having upper inputs for a mask, inhalant and oxygen. The administered medication or oxygen is trapped within the rebreathing bag resulting in almost total delivery to the lungs, through one-way valves, during inhalation. Due to the flexibility of the rebreathing bag, it deflates during inhalation indicating the extent of intake.

In amelioration of some of the known shortcomings in the art of pulmonary drug delivery, Applicant also developed a number of inhaler spacer technologies for use with different types of inhalers, as described in US patent nos. U.S. Pat. Nos. 11,207,476; 11,426,543 and 11,458,264.

In light of conventional spacer practices, Applicant has identified a shortcoming in the art of spacers, where a chamber of conventional spacers generally comprises inlets and outlets arranged with specific orientations to each other, with such relative orientations not conducive to proper dispersal of a drug aerosol from an inhaler. For example, conventional spacers comprise opposed inlets and outlets, such as that of Schleufe, or shared inlet-outlets, such as the single bidirectional channel of Sackner, Zoltan and Savoullas, or inlets and outlets laterally arranged in a single plane, such as those of Lisberg.

Additionally, conventional spacer chambers are also not optimised for efficient aerosolised drug delivery as such conventional inlets, outlets and/or spacer chambers generally present impediments to efficient aerosolised drug dispersion due to angular and abrupt surface transitions and abrupt surface terminations, which interrupts the flow of drug dispersion in the chamber and provides surfaces for an aerosolised drug to adhere to when impacting thereagainst.

The current invention was conceived with these shortcomings in mind.

### SUMMARY OF THE INVENTION

The skilled addressee is to appreciate that reference in this specification to a ‘sphere’ and derivatives thereof, such as ‘spherical’, broadly refers to a globe-like, globular or substantially globe-shaped object, which is more or less spherical, and may include a sphere, a spheroid (oblate and prolate), or any similar approximately spherical body in three-dimensional space without significant internal angles where inner surfaces comprising such a body abut.

According to an aspect of the invention there is provided pulmonary drug delivery apparatus comprising:
a spherical reservoir defining respective inlet and outlet openings through a surface thereof;
an arcuate, elongate and substantially flat spine member defining an inlet aperture therethrough at one end and an outlet aperture therethrough at another end, said arcuate spine member configured to engage complementarily about an outer surface portion of said spherical reservoir such that:
   i. the inlet aperture cooperates with the inlet opening to form an inlet port normal to the surface of the spherical reservoir; and
   ii. the outlet aperture cooperates with the outlet opening to define an outlet port tangentially arranged on the surface of the spherical reservoir;
      wherein the inlet and outlet ports are opposed and out of alignment on the spherical reservoir.

The skilled addressee is to appreciate that the specific configuration of the apparatus'spherical reservoir and spine member defining complementary inlet and outlet ports provides a reservoir for dispersion of an aerosolised drug, as well as a flow pathway largely uninterrupted by structural impediments and abrupt surface transitions and terminations, thus facilitating efficient aerosolised drug delivery from the inlet port to the outlet port.

In an embodiment, the inlet opening of the spherical reservoir comprises a circular or rounded opening normal to a centre point of the spherical reservoir.

In an embodiment, the outlet opening of the spherical reservoir comprises a slot.

In an embodiment, the outlet opening is offset from the inlet opening along the surface of the reservoir at an angle between 60° and 170°.

Typically, the slot is offset at a substantially 90° angle from the inlet opening along the surface of the reservoir.

In an embodiment, the inlet and outlet ports are coplanar on the surface of the spherical reservoir.

In an embodiment, the inlet and outlet openings are defined on a great circle or orthodrome of the spherical reservoir.

In an embodiment, the spherical reservoir is manufactured from a flexible material, such as a polymer, so that the reservoir is collapsible due to pressure fluctuations therein to accommodate a breathing pattern and/or rebreathing.

In an embodiment, the spherical reservoir is manufactured from, or internally coated with, an antistatic material to minimise adhesion of aerosolised drug particles.

In an embodiment, the spherical reservoir defines at least one engagement part on the surface thereof to facilitate complementarily engagement thereto by the spine member.

In an embodiment, the at least one engagement part comprises an engagement collar defined about a portion of the inlet and/or outlet opening, said engagement collar configured for engagement with the spine member.

In an embodiment, the spherical reservoir is configured to define a predetermined internal volume.

In an embodiment, the predetermined internal volume comprises a range of between 30 ml and 2500 ml.

In an embodiment, the spine member is configured such that the arcuation of said spine member conforms substantially to the outer surface portion arcuation of the spherical reservoir.

In an embodiment, a shape of the inlet aperture is configured to conform with a shape of the inlet opening.

In an embodiment, a shape of the outlet aperture is configured to conform with a shape of the outlet opening.

In an embodiment, the spine member defines an engagement mount about a portion of the inlet and/or outlet apertures, said engagement mount configured to mount an inlet and/or outlet adaptor, respectively.

In an embodiment, the engagement mount is configured to mount the inlet and/or outlet adaptor in a fluid-tight manner to the spherical reservoir.

In an embodiment, the apparatus includes the inlet and/or outlet adaptor.

In an embodiment, the inlet adaptor comprises an inhaler adaptor configured to arrange an inhaler in fluid-tight communication with the spherical reservoir.

In an embodiment, the inhaler is selectable from a non-exhaustive group consisting of ametered-dose inhaler (MDI), a dry powder inhaler (DPI), a soft mist inhaler and a nebuliser.

In an embodiment, the inlet port comprises an adjustable supplementary inlet port via which fluid, such as air, is able to enter the spherical reservoir.

In an embodiment, the inlet adaptor comprises the adjustable supplementary inlet port.

In an embodiment, the inlet adaptor defines a supplementary fluid passage therethrough, said supplementary fluid passage selectively adjustable to control a volume flow of fluid passable into the spherical reservoir.

In an embodiment, the inlet adaptor defines a valve cradle about the inlet port, the valve cradle defining the supplementary fluid passage, an annular valve member complementarily rotatably receivable within said valve cradle and defining a valve aperture, wherein selective rotation of the valve member within the valve cradle aligns or misaligns the supplementary fluid passage and valve aperture accordingly, whereby a volume flow of fluid passable into the spherical reservoir is infinitely adjustable.

In an embodiment, the outlet adaptor is configured to arrange the spherical reservoir in fluid communication with a respiratory passageway of a person.

In an embodiment, the outlet adaptor is selectable from a non-exhaustive group consisting of a mouthpiece and a mask facepiece.

In an embodiment, the outlet adaptor comprises a valve configured to admit fluid flow from the spherical reservoir to a respiratory passageway of a person, in use, and to divert return fluid flow from said respiratory passageway of a person to the atmosphere.

In an embodiment, the valve comprises an angled flap membrane arranged transversely across a fluid passageway of said outlet adaptor, an angle of said flap membrane facilitating admission or diversion of fluid flow. The skilled addressee is to appreciate the broad definition of a flap to comprise ‘something flat and broad that is attached at one side only and hangs loosely or covers an opening’.

In an embodiment, the angle of the flap membrane with respect to the fluid passageway of said outlet adaptor is between 30° and 90°.

In an embodiment, the angle of the flap membrane with respect to the fluid passageway of said outlet adaptor is between 30° and 60°.

In an embodiment, the valve comprises a support, such as a wire mesh, across said fluid passageway for supporting the flap membrane across the fluid passageway during diversion of fluid flow.

In an embodiment, the valve comprises a trapdoor flap via which return fluid flow from said respiratory passageway of a person is diverted to the atmosphere.

In an embodiment, the inlet adaptor and/or outlet adaptor comprises a whistle configured to provide auditory feedback on an inspiration and/or expiration rate when using the

In an embodiment, the inlet adaptor comprises a handle to facilitate holding of the apparatus during use thereof.

According to a further aspect of the invention there is provided pulmonary drug delivery apparatus, substantially as herein described and/or illustrated.

### BRIEF DESCRIPTION OF THE DRAWINGS

The description will be made with reference to the accompanying drawings in which:

FIG. 1 is a diagrammatic perspective-view rendered representation of one embodiment of pulmonary drug delivery apparatus, in accordance with an aspect of the present invention;

FIGS. 2a and 2b show diagrammatic representations of substantially unimpeded aerosolised drug pathways through the pulmonary drug delivery apparatus of FIG. 1;

FIG. 3 is a diagrammatic perspective-view wireframe representation of an embodiment of the pulmonary drug delivery apparatus of FIG. 1;

FIG. 4 is a diagrammatic exploded view representation of the pulmonary drug delivery apparatus of FIG. 3;

FIG. 5 is a diagrammatic perspective-view wireframe representation of the spherical reservoir and spine member of the pulmonary drug delivery apparatus of FIG. 3;

### FIG. 6 is a diagrammatic exploded perspective-view wireframe representation of the spherical reservoir, spine member, inlet adaptor and outlet adaptor of the pulmonary drug

FIG. 7 is a diagrammatic wireframe representation of examples of inlet adaptors with a nebuliser inhaler shown;

### FIG. 8 is a diagrammatic exploded wireframe representation of the outlet adaptor showing an example of a valve;

FIGS. 9 to 12 are diagrammatic perspective-view representations of different examples of the outlet adaptor with a valve;

FIG. 13 is a diagrammatic exploded perspective-view rendered representation of examples of constituent parts of pulmonary drug delivery apparatus according to aspects of the present invention;

FIG. 14 is a diagrammatic perspective-view representation of a further embodiment of a pulmonary drug delivery apparatus, in accordance with aspects of the present invention;

FIG. 15 is a diagrammatic exploded representation of the pulmonary drug delivery apparatus of FIG. 14;

FIG. 16 is a diagrammatic top perspective-view representation of the pulmonary drug delivery apparatus of FIG. 14; and

FIG. 17 shows an exploded perspective-view representation of one embodiment of adjustable supplementary inlet port for the pulmonary drug delivery apparatus.

### DETAILED DESCRIPTION OF EMBODIMENTS

Further features of the present invention are more fully described in the following description of several non-limiting embodiments thereof. This description is included solely for the purposes of exemplifying the present invention to the skilled addressee. It should not be understood as a restriction on the broad summary, disclosure or description of the invention as set out above.

In the figures, incorporated to illustrate features of the example embodiment or embodiments, like reference numerals are used to identify like parts throughout. Additionally, features, mechanisms and aspects well-known and understood in the art will not be described in detail, as such features, mechanisms and aspects will be within the understanding of the skilled addressee.

Additionally, the accompanying figures do not represent engineering or design drawings, but provide a functional overview of the invention only. As a result, features and practical construction details required for various embodiments may not be indicated in each figure, but such construction requirements will be within the understanding of the skilled addressee.

Broadly, the present invention provides for pulmonary drug delivery apparatus 10 which typically acts as an inhaler spacer for aerosolised drug dispersion and delivery between an inhaler 38 and a respiratory passageway of a person. Apparatus 10 has been devised to be particularly conducive to proper collection and dispersal of a drug aerosol from an inhaler 38, whilst further facilitating ease and efficiency of aerosolised drug delivery to a person. Importantly, such collection, dispersal and delivery are suitable for variable breathing patterns and associated rebreathing of a patient, as required.

Additionally, as described in more detail below, different types of inhalers 38, as well as different means of aerosolised drug output, albeit via a mouthpiece, a facepiece, extension tube, or the like. Accordingly, in one embodiment, the inhaler 38 may comprise a metered-dose inhaler (MDI), a dry powder inhaler (DPI), a soft mist inhaler, a nebuliser, or the like. Similarly, the output means, typically via the output adaptor 36 described below, may comprise a mouthpiece, a mask facepiece, an extension tube, or the like.

With reference now to the accompanying figures, there is exemplified embodiments of such pulmonary drug delivery apparatus 10. In one embodiment, apparatus 10 broadly comprises a spherical reservoir 10 and a spine member 20, as shown. The skilled addressee is to appreciate that reference to ‘spherical’ includes reference to shapes that are more or less spherical, such as a sphere, a spheroid, or any similar approximately spherical body in three-dimensional space without significant internal angles where inner surfaces comprising such a body abut. For example, the spherical reservoir 10 may comprise an oblong spheroid such as a football-shape, or the like.

The spherical reservoir 12 generally defines respective inlet and outlet openings 14 and 16 through a surface 18 thereof. In one embodiment, the inlet opening 14 of the spherical reservoir 12 comprises a circular or oval opening normal to a centre point of the spherical reservoir 12. For example, the inlet opening comprises a round or oval opening on a side of the spherical reservoir 12 with said opening directed or angled onto a centre point of the spherical reservoir 12, as shown.

In one embodiment, the outlet opening 16 of the spherical reservoir 12 comprises a slot, but of course variations hereon are possible and expected. In various embodiment, the outlet opening 16 may be offset from the inlet opening 14 along the surface 18 of the reservoir at an angle between 60° and 170°. Typically, such a slot is offset at a substantially 90° angle from the inlet opening 14 along the surface 18 of the reservoir 12.

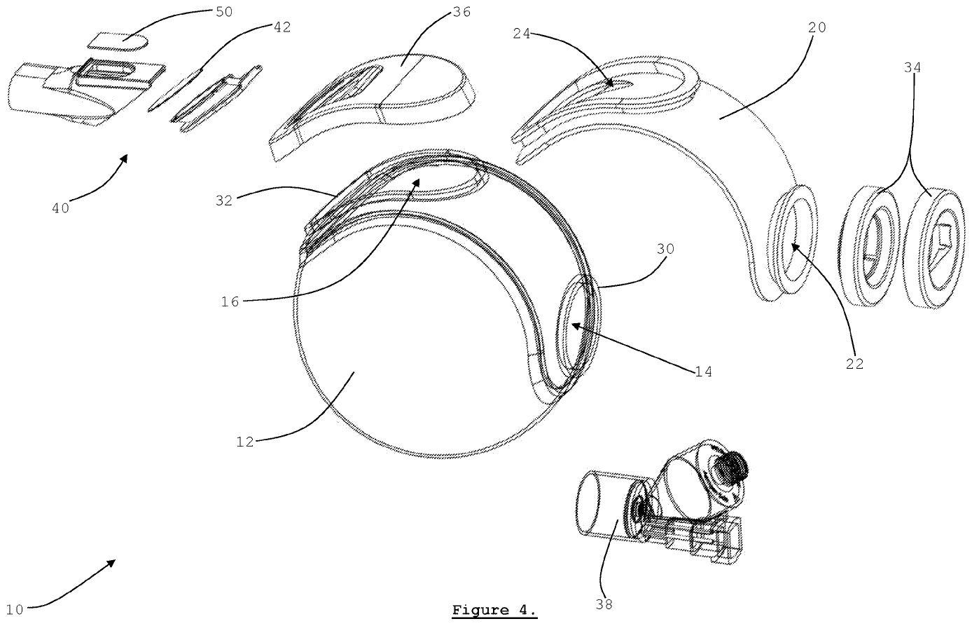

For example, as seen in FIG. 4, the inlet opening is on a side of the reservoir 12, with the outlet opening defined roughly a quarter revolution about the spherical reservoir 12. In a typical embodiment, the inlet and outlet openings 14 and 16 are defined on a great circle or orthodrome of the spherical reservoir 12, but variations hereon are possible and anticipated. With the outlet opening 14 being a slot allows a tangential arrangement of the outlet port 28. Such a configuration is important, as it facilitates the inlet and outlet ports 26 and 28 being opposed, as well as out of alignment, whilst being generally coplanar on the spherical reservoir 12, as described below.

In one embodiment, the spherical reservoir 12 is manufactured from a flexible material, such as a polymer, e.g. silicone, so that the reservoir 12 is collapsible due to pressure fluctuations therein to accommodate a breathing pattern and/or rebreathing of a person using apparatus 10. In one embodiment, the spherical reservoir 12 is manufactured from, or internally coated with, an antistatic material to minimise adhesion of aerosolised drug particles. For example, an internal surface of the reservoir 12 may be coated with an antistatic agent, or subjected to an ultra-violet light curing process to reduce static electrical activity and promote hydrophobic properties for reducing adherence of liquids to the material, and/or the like.

In one embodiment, the spherical reservoir 12 defines at least one engagement part 30 on the surface 18 thereof to facilitate complementarily engagement thereto by the spine member 20. For example, in the exemplified embodiment, the at least one engagement part 30 comprises an engagement collar defined about a portion of the inlet and outlet openings 14 and 16, as shown, with said engagement collar configured for engagement with the spine member 20. Such engagement may include an interference fit, or the like.

For example, the spine member 20 may be manufactured from a substantially rigid polymer material, with the reservoir 12 manufactured from a flexible silicone material. The spine member 20 may then define suitable receptacles, such as peripheral grooves etc. for receiving the engagement part(s) 30 of the reservoir therein, typically as an interference fit to allow removal and replacement of the reservoir 12 as required.

In one embodiment, the spherical reservoir 12 is configured to define a predetermined internal volume. In an embodiment, the predetermined internal volume comprises a range of between 30 ml and 2500 ml. Typically, such a predetermined internal volume is selectable according to a treatment requirement of a person, generally to accommodate breathing pattern, rebreathing capability, lung capacity, or the like. As a result, variations on this internal volume are possible and expected.

The spine member 20 generally comprises an elongate, arcuate and substantially flat member, as shown. The spine member 20 defines an inlet aperture 22 therethrough at one end, as well as an outlet aperture 24 therethrough at the other end. The spine member 20 is configured to engage complementarily about an outer surface portion of the spherical reservoir 12 such that the inlet aperture 22 cooperates with the inlet opening 14 to form an inlet port 26 normal to the surface 18 of the spherical reservoir 12. Similarly, the outlet aperture 24 cooperates with the outlet opening 16 to define an outlet port 28 which is tangentially arranged on the surface 18 of the spherical reservoir 12.

The spine member 20 is typically configured such that the arcuation of said spine member 20 conforms substantially to the outer surface portion arcuation of the spherical reservoir 12, as shown. Such a configuration provides a close fit between the reservoir 12 and spine member 20 where the spine member 20 lies against the reservoir 12. The spine member 20 may include suitable fluid seals at the inlet and outlet apertures 22 and 24 to facilitate fluid-tight engagement with the spherical reservoir 12, or the like.

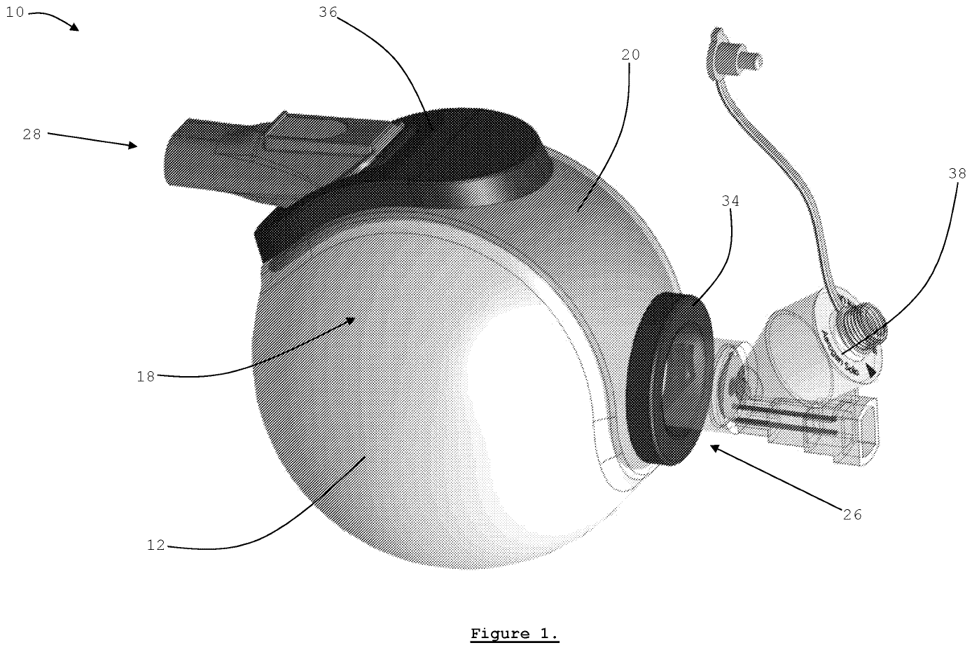

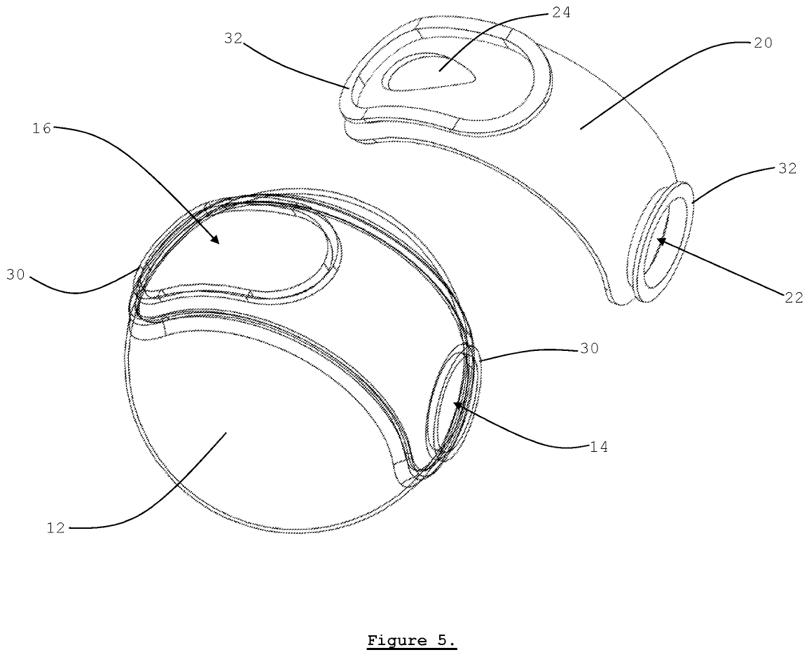

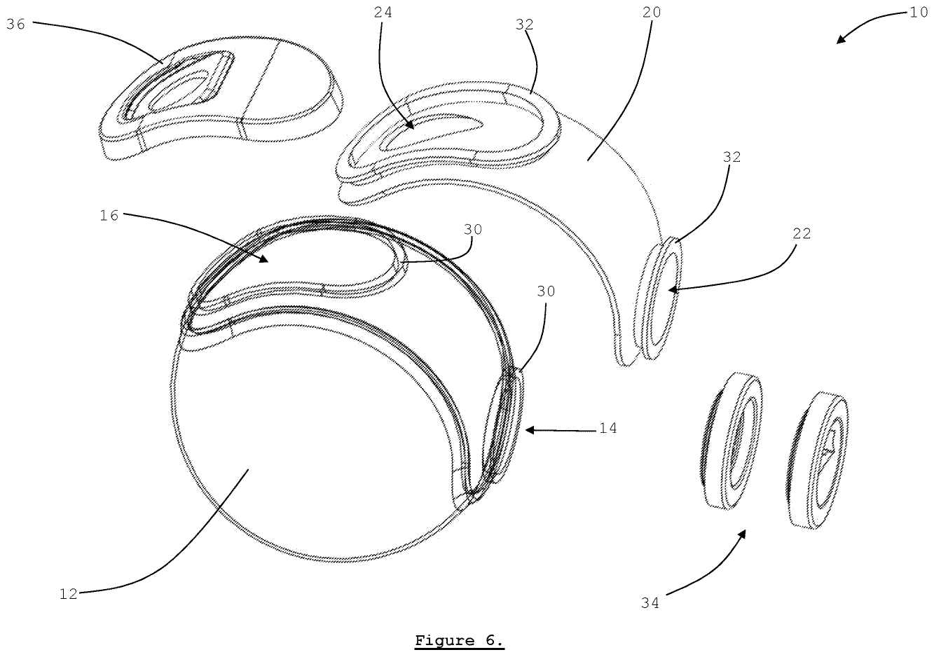

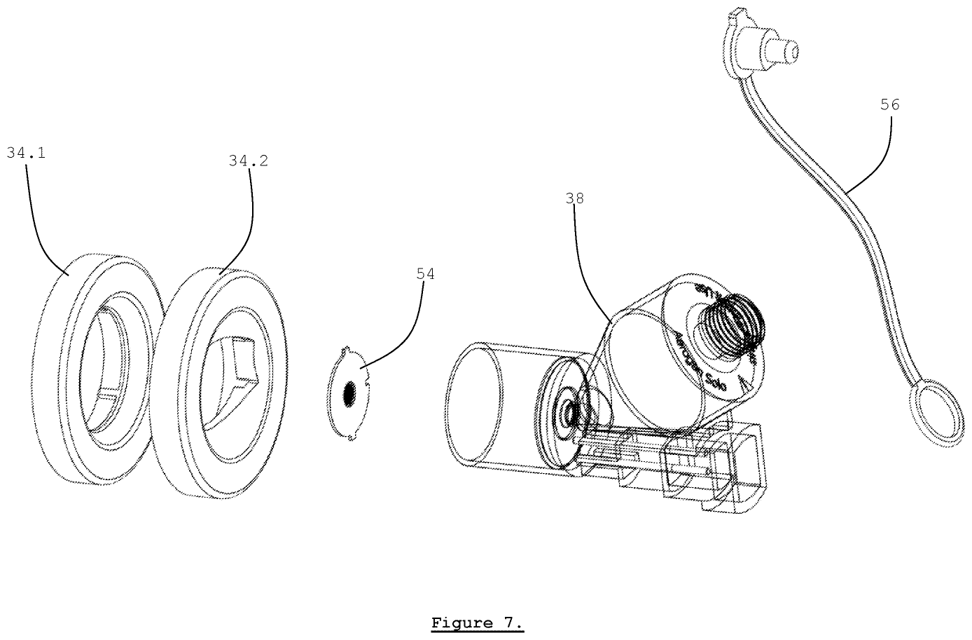

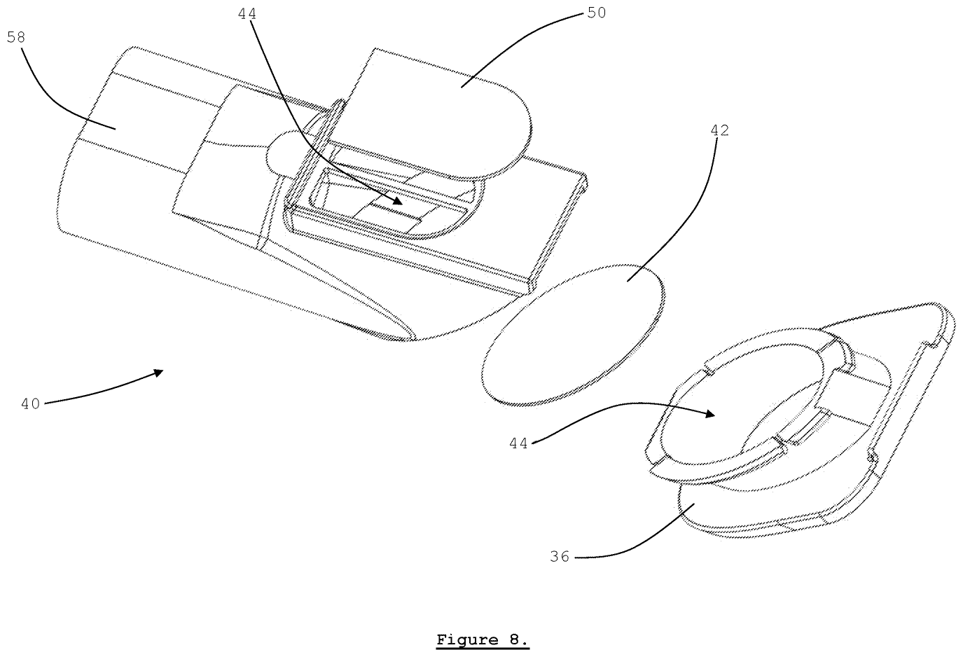

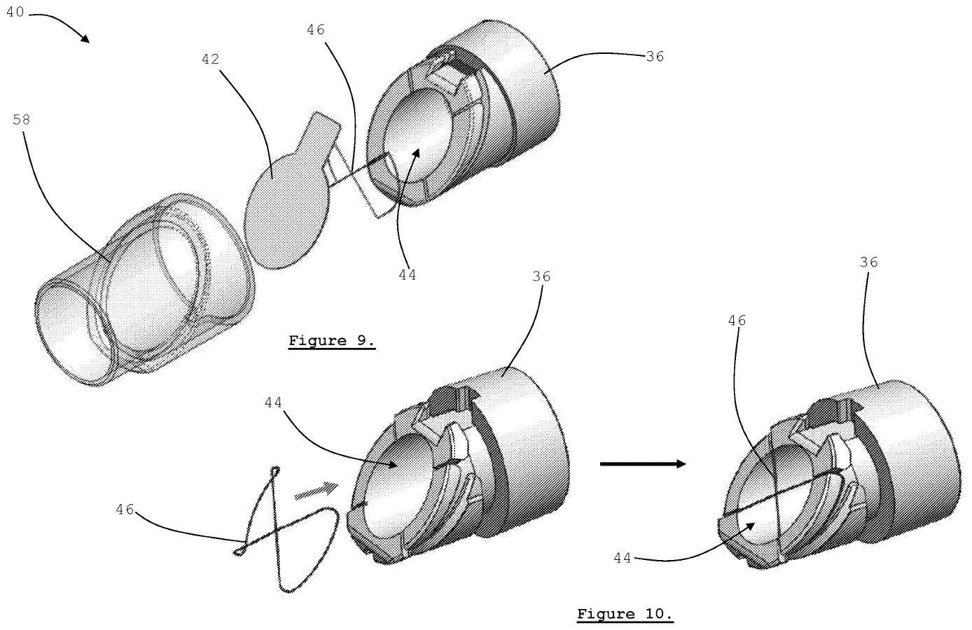

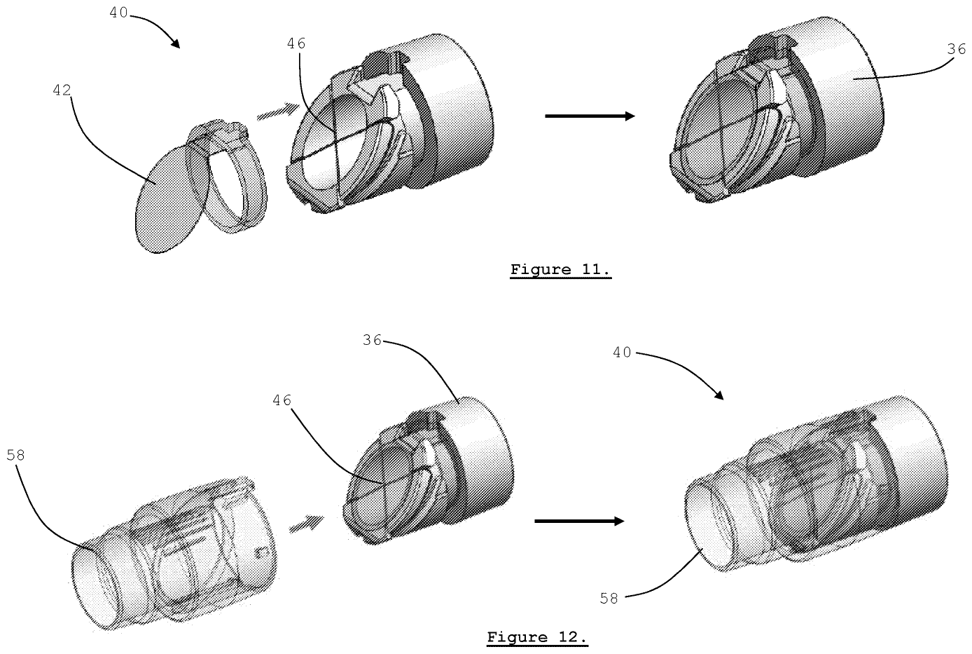

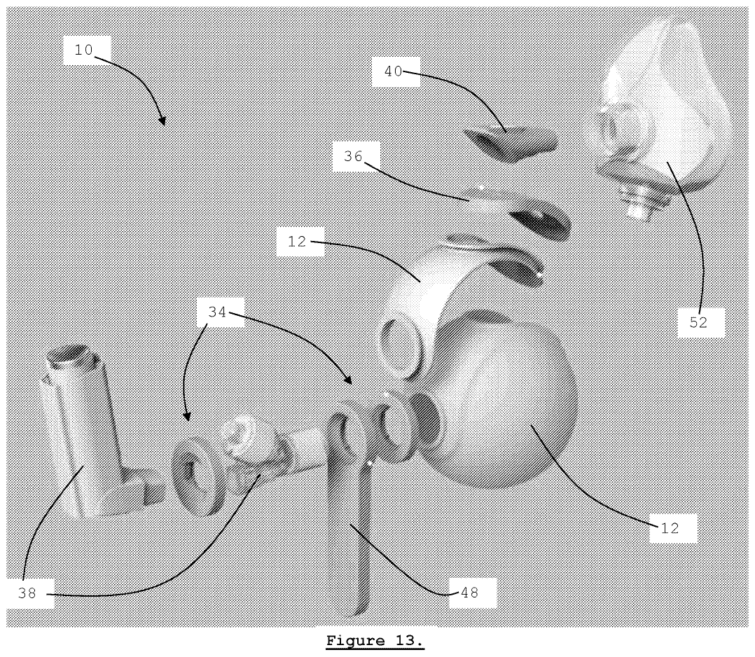

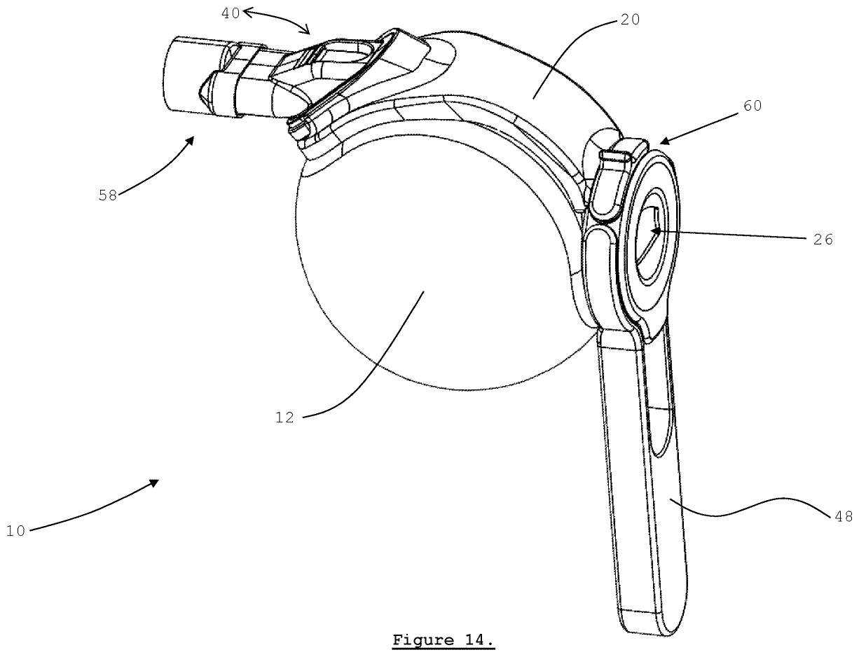

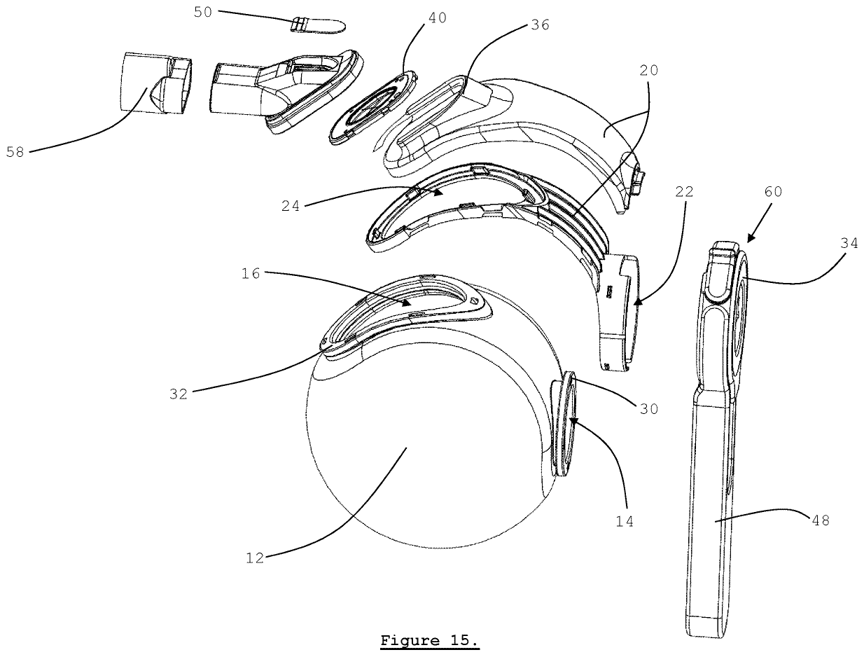

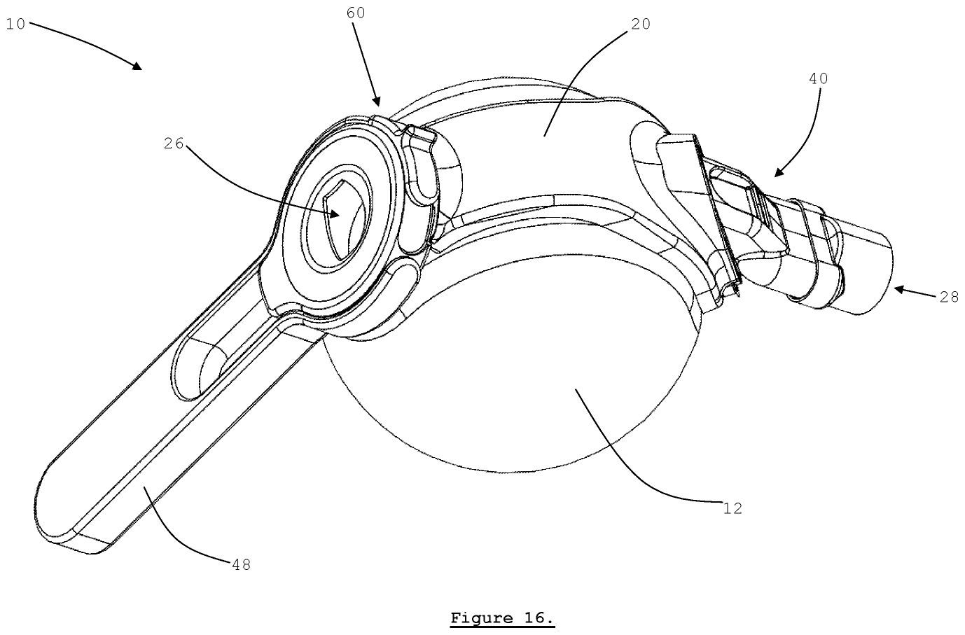

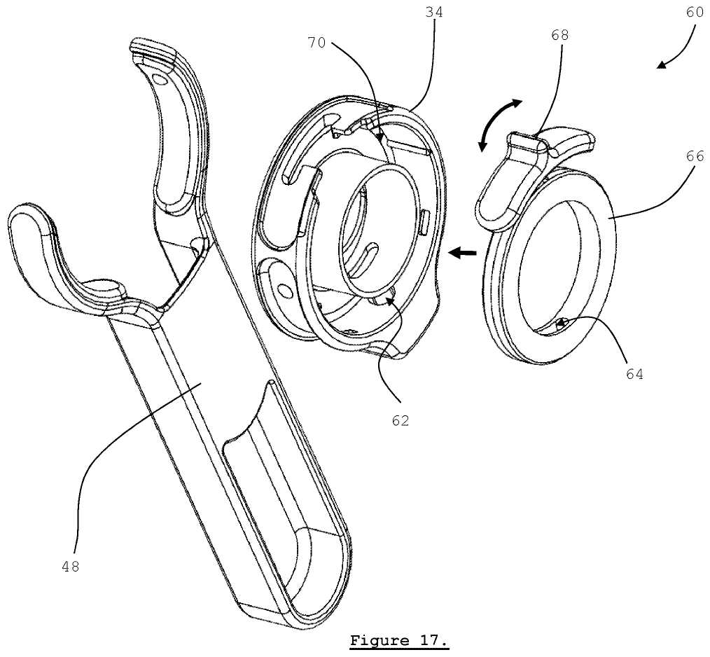

the spine member 20 may be realised in various functional configurations. For example, the embodiment of FIGS. 1 to 13 show spine member as a unitary component, whereas FIGS. 14 to 16 show an embodiment where the spine member 20 is comprised of two interlocking parts. Such variations may facilitate different manufacturing methods and are included within the scope of the present disclosure.

In one embodiment, a shape of the inlet aperture 22 is configured to conform with a shape of the inlet opening 14. Similarly, in one embodiment, a shape of the outlet aperture 24 is configured to conform with a shape of the outlet opening 16. Such conformity between openings and apertures generally facilitates cooperation between the reservoir 12 and spine member 20 to defines the inlet and outlet ports 26 and 28.

Typically, apparatus 10 includes inlet and outlet adaptors 34 and 36 in order to provide modularity for use with different inhalers 38, as well as output means, as mentioned above. In one embodiment, the spine member 20 defines an engagement mount 32 about a portion of the inlet and/or outlet apertures 22 and 24, as shown, with said engagement mount 32 configured to mount an inlet and/or outlet adaptor 34 and 36, respectively. Typically, the engagement mount 32 is configured to mount the inlet and outlet adaptors 34 and 36 in a fluid-tight manner to the spherical reservoir 12. Of course, in other embodiments, the inlet and/or outlet adaptor 34 and 36 may also be included as part of the spine member 20 in a unitary manner, or the like.

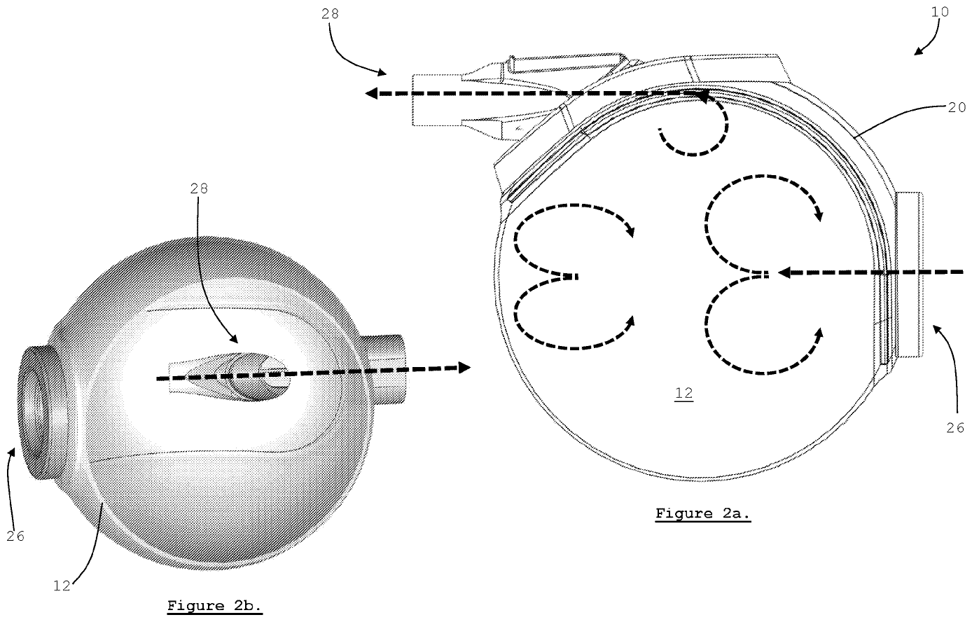

In the manner described, the resulting inlet and outlet ports 26 and 28 are opposed, out of alignment and generally, but not necessarily, coplanar on the spherical reservoir 12. The ports 26 and 28 are opposed in the fluid flow therethrough are generally directed in opposite directions, as shown in FIG. 2a. Additionally, the ports 26 and 28 are out of alignment in that they do not align directly opposite each other on the reservoir 12. Furthermore, the ports 26 and 28 are typically coplanar on the reservoir in that they lie in a shared plane, as defined by the spine member 30, relative to the reservoir 12, but this is optional.

The skilled addressee is to appreciate that the specific configuration of the apparatus'spherical reservoir 12 and spine member 30 defining complementary inlet and outlet ports 26 and 28 provides a reservoir 12 for dispersion of an aerosolised drug, as well as a flow pathway largely uninterrupted by structural impediments and abrupt surface transitions and terminations, thus facilitating efficient aerosolised drug delivery from the inlet port 26 to the outlet port 28.

For example, with specific reference to FIG. 2, such flow pathway is indicated via dashed arrows. In FIG. 2a, the inlet port 26 facilitates direct entry of an aerosolised drug from the inhaler 38 into the reservoir 12, generally directed at a centre point of the spherical reservoir 12, as shown, due to the specific configuration of the inlet opening and inlet aperture cooperating to form inlet port 26. In contrast, the outlet port 18, lying tangentially on the surface of the reservoir, as shown more clearly in the sectioned view of FIG. 2b, due to the cooperation and specific configuration of the outlet opening 16 and outlet aperture 24, ensures that the inlet and outlet ports 26 and 28 are opposed, out of alignment and typically, but not necessarily, coplanar on the spherical reservoir 12.

Importantly, such qualities of the ports 26 and 28 being opposed, out of alignment and typically coplanar on the spherical reservoir 12 allows an aerosolised drug to enter the reservoir and disperse until a person is ready to inhale, without impacting against any angular and abrupt surface transitions and abrupt surface terminations, given the ‘smooth’ inner arrangement of the spherical reservoir, and the relative ‘smooth’ and unobstructed exit flow pathway provided by the tangentially-arranged outlet port 28 further facilitates unimpeded exit of such dispersed aerosolised drug from the reservoir 12. The spherical nature of the reservoir 12 facilitates dispersal of aerosolised drug and establishment of eddy flow within the reservoir, as shown in FIG. 2a, with such eddy flow paths easily transitioned into tangentially-arranged outlet port 28. In this manner, proper drug dispersal is improved, as well as efficient delivery of such dispersed aerosolised drug to a person.

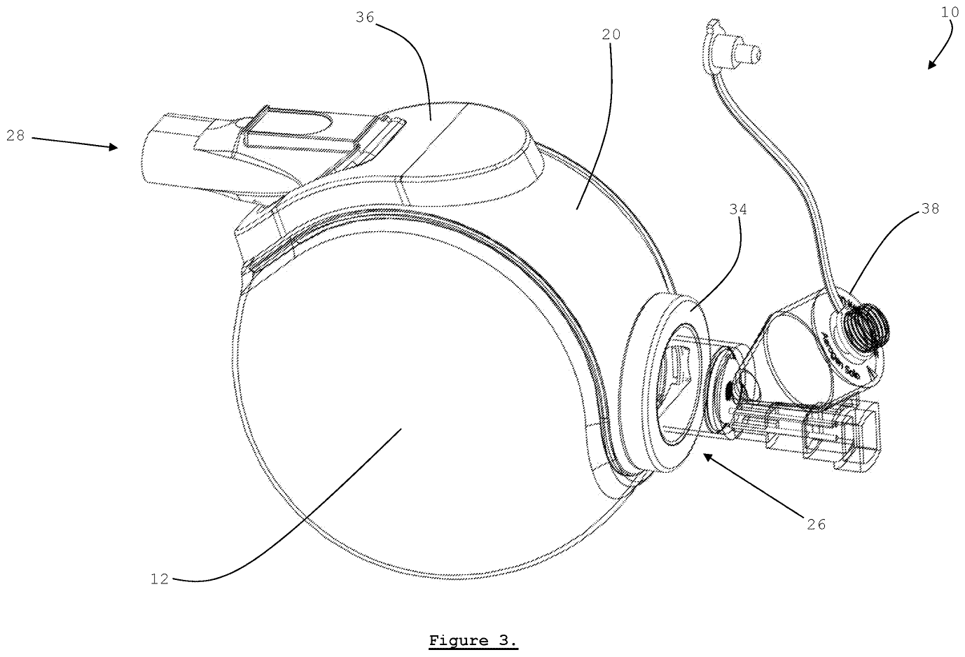

In one embodiment, the inlet adaptor 34 is configured to arrange the inhaler 38 in fluid-tight communication with the spherical reservoir 12. As mentioned, such inlet adaptor 34 may take various forms to cater for different types of inhalers 38, such as MDI, DPI, nebulisers, etc. Depending on inhaler 38 used, supporting features such as seals, support plates, closing caps, etc. may be relevant. For example, in the embodiment of FIGS. 3 and 7, the inhaler 38 comprises a nebuliser with a support plate/seal 54 and a closing cap 56. Variations hereon are possible and expected.

In one embodiment, the inlet port 26 comprises an adjustable supplementary inlet port 60 via which fluid, such as air, is able to enter the spherical reservoir 12. Such an adjustable supplementary inlet port 60 may facilitate a person inhaling under circumstances where the spherical reservoir 12 has collapsed due to inhalation pressure, or the like. The adjustable supplementary inlet port 60 may also be selectively activatable, such as an aperture which may be closed by a person's finger until required, or the like.

In one embodiment, an example of which is shown in more detail in FIG. 17, the the inlet adaptor 34, which forms part of the overall inlet port 26, comprises the adjustable supplementary inlet port 60. In the exemplified embodiment, the inlet adaptor 34 defines a supplementary fluid passage 62 therethrough, as shown, with said supplementary fluid passage 62 selectively adjustable to control a volume flow of fluid passable into the spherical reservoir 12 during use of apparatus 10.

In the exemplified embodiment, the inlet adaptor 34 defines a valve cradle 70 about the inlet port 26, with this valve cradle 70 defining the supplementary fluid passage 62, as shown. An annular valve member 66 is complementarily rotatably receivable within said valve cradle 70, with the valve member 66 further defining a valve aperture 64, as shown. In this manner, selective rotation of the valve member 66 within the valve cradle 70, typically via external adjustment knob 68, aligns or misaligns the supplementary fluid passage 62 and valve aperture 64 accordingly, whereby a volume flow of fluid passable into the spherical reservoir 12 is infinitely adjustable.

In such an embodiment, the adjustable supplementary inlet port 60 serves as an additional, but separate, channel from the overall inlet port 26 which is generally occupied entirely by the inhaler 38, i.e. in a fluid tight manner. This separate adjustable supplementary inlet port 60 is useful for providing a passage for any additional air to flow into the reservoir 12, such as when the reservoir 12 is fully collapsed and a user is not suddenly denied suitable air for breathing. The separate fluid passage 62 is further able to provide a channel for entrained air to be drawn through into the reservoir 12 while the reservoir 12 slowly re-expands with its own elastic recoil, i.e. facilitates with inhalation of additional air such as where the spherical reservoir has collapsed before the user has completed inhaling, and to allow air to flow into the reservoir as it re-expands by its own elastic recoil. Additionally, the separate fluid passage 62 may also provide a connection point for oxygen tubing or the like to be attached for supply of supplemental oxygen to a patient if needed during administration of pulmonary drug delivery.

In one embodiment, the outlet adaptor 36 is generally configured to arrange the spherical reservoir 12 in fluid communication with a respiratory passageway of a person. In one embodiment, the outlet adaptor 36 may engage with a mouthpiece 58, a mask facepiece 58, or the like. Again, variations hereon are possible and expected.

In a typical embodiment, the outlet adaptor 36 further comprises a valve 40 which is configured to admit fluid flow from the spherical reservoir 12 to a respiratory passageway of a person, in use, i.e. prevent return fluid flow from the person's respiratory passageway returning to the reservoir, but to instead divert return fluid flow from said respiratory passageway of a person to the atmosphere. As shown by the various examples in FIGS. 8 to 12, one embodiment of the valve 40 typically comprises an angled flap membrane 42 which is arranged transversely across a fluid passageway 44 of the outlet adaptor 36.

Importantly, in one embodiment an angle of said flap membrane 42 typically facilitates admission or diversion of fluid flow, as it minimises any impediment of fluid flow from the reservoir and inversely to the atmosphere, i.e. the angled flap 42 presents a ‘smooth’ impediment to desired fluid flow. Accordingly, the angle of the flap membrane 42 with respect to the fluid passageway 44 of said outlet adaptor is generally between 30° and 60°. Similarly, the flap membrane 42 is typically selected and configured to be lightweight and easily flexible in order to offer low resistance to being opened in order to reduce creation of turbulence by disturbing fluid flow through the valve, thereby maximising efficient pulmonary drug delivery. For example, a film-like, fluid-tight flap membrane with very little mass, or the like.

In one embodiment, the valve 40 comprises a wire mesh 46 arranged across said fluid passageway 44 for supporting the flap membrane 42 across the fluid passageway 44 during diversion of fluid flow. In one embodiment, the valve 40 also comprises a trapdoor flap 50 via which return fluid flow from said respiratory passageway of a person is diverted to the atmosphere. The valve 40 typically includes an exit passageway, often part of the fluid passageway 44, which is closed by said trapdoor flap 50 when fluid passes over the flap membrane 42 to the respiratory passageway of a person. If fluid is passed from the respiratory passageway of a person into the passageway 44, the flap membrane 42 is pushed against the wire mesh 46 to seal entry to the reservoir and the flap membrane 42 angles any such returning fluid towards the trapdoor flap 50, via the exit passageway, where said returning fluid is expelled to the

In one embodiment, the inlet adaptor 34 and/or outlet adaptor 36 comprises a whistle (not shown) which is configured to provide auditory feedback on an inspiration and/or expiration rate when using the apparatus 10. In one embodiment, the inlet adaptor 34 comprises a handle 48 to facilitate holding of the apparatus 10 during use thereof.

Applicant believes it particularly advantageous that the present invention provides for apparatus 10 having specific configurations facilitating proper dispersal of a drug aerosol from an inhaler, whilst presenting minimum impediment to efficient aerosolised drug dispersion due to angular surface transitions and abrupt surface terminations. The specific arrangement of the inlet and outlet ports 26 and 28 also facilitates efficiency of aerosolised drug delivery through apparatus 10.

Applicant also believe it advantageous that the specific configurations of, and interactions between, the adjustable supplementary inlet port 60, reservoir 12 and valve 40 facilitate efficient dispersion and delivery of an aerosolised drug without providing unnecessary impediment to fluid flow. In addition, collection and dispersal of a drug aerosol from an inhaler via apparatus 10 is not adversely affected by the variability of breathing patterns between patients, whereas this variability in breathing patterns may have adverse effects on performance of conventional aerosolised drug delivery devices.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. As used herein, the term “and/or” includes any and all combinations of one or more of the associated listed items. No language in the specification should be construed as indicating any non-claimed subject matter as essential to the practice of the claimed subject matter. Spatially relative terms, such as “inner,” “outer,” “beneath,” “below,” “lower”, “above,” “upper,” and the like, may be used herein for ease of description to describe one element or feature's relationship to another element(s) or feature(s) as illustrated in the figures.

It is also to be appreciated that reference to “one example” or “an example” of the invention, or similar exemplary language (e.g., “such as”) herein, is not made in an exclusive sense. Accordingly, one example may exemplify certain aspects of the invention, whilst other aspects are exemplified in a different example. Variations, modifications and/or enhancements of one or more embodiments described herein might become apparent to those of ordinary skill in the art upon reading this application. The inventor(s) expects skilled artisans to employ such variations as appropriate, and the inventor(s) intends for the claimed subject matter to be practiced other than as specifically described herein.

1. A pulmonary drug delivery apparatus comprising:
a spherical reservoir defining respective inlet and outlet openings through a surface thereof, and
an arcuate, elongate and substantially flat spine member defining an inlet aperture therethrough at one end and an outlet aperture therethrough at another end, said arcuate spine member configured to engage complementarily about an outer surface portion of said spherical reservoir such that:
the inlet aperture cooperates with the inlet opening to form an inlet port normal to the surface of the spherical reservoir; and
the outlet aperture cooperates with the outlet opening to define an outlet port tangentially arranged on the surface of the spherical reservoir;
wherein the inlet and outlet ports are opposed and out of alignment on the spherical reservoir.

2. Apparatus of claim 1, wherein the inlet opening of the spherical reservoir comprises a circular or rounded opening normal to a centre point of the spherical reservoir.

3. Apparatus of claim 1, wherein the outlet opening of the spherical reservoir comprises a slot.

4. Apparatus of claim 1, wherein the outlet opening is offset from the inlet opening along the surface of the reservoir at an angle of between 60° and 170° relative to a centre point of said reservoir.

5. Apparatus of claim 1, wherein the outlet opening is offset at a substantially 90° angle from the inlet opening along the surface of the reservoir relative to a centre point of said reservoir.

6. Apparatus of claim 1, wherein the inlet and outlet ports are coplanar relative to the spherical reservoir.

7. Apparatus of claim 1, wherein the inlet and outlet openings are defined on a great circle or orthodrome of the spherical reservoir.

8. Apparatus of claim 1, wherein the spherical reservoir is manufactured from a flexible material, such as a polymer, so that the reservoir is collapsible due to pressure fluctuations therein to accommodate a breathing pattern and/or rebreathing.

9. Apparatus of claim 1, wherein the spherical reservoir is manufactured from, or internally coated with, an antistatic material to minimise adhesion of aerosolised drug particles.

10. Apparatus of claim 1, wherein the spherical reservoir defines at least one engagement part on the surface thereof to facilitate complementarily engagement thereto by the spine member wherein the at least one engagement part comprises an engagement collar defined about a portion of the inlet and/or outlet opening, said engagement collar configured for engagement with the spine member.

11.-13. (canceled)

14. Apparatus of claim 1, wherein the spine member is configured such that the arcuation of said spine member conforms substantially to the outer surface portion arcuation of the spherical reservoir.

15.-16. (canceled)

17. Apparatus of claim 1, wherein the spine member defines an engagement mount about a portion of the inlet and/or outlet apertures, said engagement mount configured to mount an inlet and/or outlet adaptor, respectively and wherein the engagement mount is configured to mount the inlet and/or outlet adaptor in a fluid-tight manner to the spherical reservoir.

18.-19. (canceled)

20. Apparatus of claim 17, wherein the inlet adaptor comprises an inhaler adaptor configured to arrange an inhaler in fluid-tight communication with the spherical reservoir and wherein the inhaler is selectable from a group consisting of a metered-dose inhaler (MDI), a dry powder inhaler (DPI), a soft mist inhaler and a nebuliser.

21.-24. (canceled)

25. Apparatus of claim 20, wherein the inlet adaptor defines a supplementary fluid passage therethrough, said supplementary fluid passage is selectively adjustable to control a volume flow of fluid passable into the spherical reservoir.

26. Apparatus of claim 25, wherein the inlet adaptor defines a valve cradle about the inlet port, the valve cradle defining the supplementary fluid passage, an annular valve member complementarily rotatably receivable within said valve cradle and defining a valve aperture, wherein selective rotation of the valve member within the valve cradle aligns or misaligns the supplementary fluid passage and valve aperture accordingly, whereby a volume flow of fluid passable into the spherical reservoir is adjustable via the valve aperture being infinitely adjustable.

27. Apparatus of claim 17, wherein the outlet adaptor is selectable from a group consisting of a mouthpiece and a mask facepiece and configured to arrange the spherical reservoir in fluid communication with a respiratory passageway of a person.

28. (canceled)

29. Apparatus of claim 17, wherein the outlet adaptor comprises a valve configured to admit fluid flow from the spherical reservoir to a respiratory passageway of a person, in use, and to divert return fluid flow from said respiratory passageway of a person to an atmosphere.

30. Apparatus of claim 29, wherein the valve comprises an angled flap membrane arranged transversely across a fluid passageway of said outlet adaptor, an angle of said flap membrane facilitating admission or diversion of fluid flow wherein the angle of the flap membrane with respect to the fluid passageway of said outlet adaptor is between 30° and 90°, and preferably between 30° and 60;
wherein the valve comprises a support across said fluid passageway for supporting the flap membrane across the fluid passageway during diversion of fluid flow as a trapdoor flap via which return fluid flow from said respiratory passageway of a person is diverted to the atmosphere.

31.-33 (canceled)

34. Apparatus of claim 17, wherein the inlet adaptor and/or outlet adaptor comprises a whistle configured to provide auditory feedback on an inspiration and/or expiration rate when using the apparatus.

35. Apparatus of claim 17, wherein the inlet adaptor comprises a handle to facilitate holding of the apparatus during use thereof.
